(19) United States
(12) Patent Application Publication
(10) Pub. No.: US 2017/0086678 A1
(43) Pub. Date: Mar. 30, 2017

(54) APPARATUS

(71) Applicant: CANON KABUSHIKI KAISHA, Tokyo (JP)

(72) Inventor: Takuji Oishi, Kawasaki-shi (JP)

(21) Appl. No.: 15/267,216

(22) Filed: Sep. 16, 2016

(30) Foreign Application Priority Data
Sep. 29, 2015 (JP) 2015-191578

(51) Int. Cl. A61B 5/00; G01N 29/24; G01N 29/44; G01N 29/04

(52) U.S. Cl. CPC A61B 5/0095; G01N 29/043; G01N 29/2418; G01N 29/4463; A61B 5/7225; G01N 2291/02475

(57) ABSTRACT

An apparatus includes a plurality of acoustic detectors detecting an acoustic wave from an object and outputting an electric signal, including a first and a second acoustic detectors each having a first and a second frequency sensitivity characteristics to output a first and a second electric signals, the first and the second frequency sensitivity characteristics are different, a memory holding a correction function created based on a third frequency sensitivity characteristics obtained from the first and the second frequency sensitivity characteristics, and an information processor correcting the first and the second electric signals using the correction function, thereby acquiring corrected electric signals and generating image data representing object information.

### BACKGROUND OF THE INVENTION

Field of the Invention

The present invention relates to an apparatus that acquires object information based on an acoustic wave generated due to a photoacoustic effect.

Description of the Related Art

In recent years, every effort has been made to develop an apparatus that adopts photoacoustic tomography (PAT) to acquire biological function information using light and acoustic waves and that is a type of apparatus that can image the interior of an living organism in a noninvasive manner.

The photoacoustic tomography is a technique involving irradiating an object with pulsed light emitted from a light source and imaging an internal tissue that is an absorber in the living organism using a photoacoustic effect in which light propagating and diffusing through the object is absorbed to generate an acoustic wave (photoacoustic wave). A temporal variation in the received acoustic wave is detected in a plurality of areas. The resultant signals are mathematically analyzed, that is, reconstructed. Thus, information concerning optical characteristics such as an absorption coefficient for the interior of the object is three-dimensionally visualized. The reconstruction is a process involving time series signals obtained at different positions are converted into distances with a propagation velocity (sound velocity) of acoustic waves and superimposing the resultant data on a space to visualize a space distribution.

Near infrared light has the property of being likely to pass through water, which forms most of the living organism, and to be absorbed by hemoglobin in the blood. Thus, using near infrared light as pulsed light allows the blood in the living organism to be imaged. Blood vessel images obtained using pulsed light with different wavelengths are compared with one another to allow measurement of oxygen saturation in the blood, which is function information. Blood around a malignant tumor is expected to have lower oxygen saturation than blood around a benign tumor. Consequently, it is expected that whether the tumor is benign or malignant can be determined based on the measured oxygen saturation.

Upon detecting a photoacoustic wave, an acoustic detector outputs an electric signal (photoacoustic signal). When the axis of ordinate represents sound pressure and the axis of abscissas represents time, a photoacoustic signal derived from an absorber is typically shaped like the letter N. The width of the photoacoustic signal in the time direction depends on the size of the absorber. A photoacoustic signal with a large signal width contains many low frequency components, whereas a photoacoustic signal with a small signal width contains many high frequency components. Therefore, a dominant frequency component in the photoacoustic signal varies according to the size of the absorber.

On the other hand, the acoustic detector is sensitive usually in a limited frequency band. Therefore, the sensitivity of the acoustic detector may be insufficient in the frequency band of the photoacoustic signal, which is dictated according to the size of the absorber. In this case, in a reconstructed image, only a contour portion is rendered. In contrast, an image of an absorber smaller than the absorber size suitable for the sensitivity of the acoustic detector is blurred.

However, predetermining the sizes of absorbers in the living organism is difficult. Thus, the frequency band in which the acoustic detector is sensitive (sensitive frequency band) needs to be widened as much as possible. In Geng Ku, et. al., “Multiple-bandwidth photoacoustic tomography”, PHYSICS IN MEDICINE AND BIOLOGY 49 (2004) 1329 1338, a plurality of acoustic detectors sensitive in different frequency bands are used to detect acoustic waves at a plurality of positions in order to virtually widen the sensitive frequency band.

Non Patent Literature 1: Geng Ku, et. al., “Multiple-bandwidth photoacoustic tomography”, PHYSICS IN MEDICINE AND BIOLOGY 49 (2004) 1329 1338

### SUMMARY OF THE INVENTION

In Geng Ku, et. al., “Multiple-bandwidth photoacoustic tomography”, PHYSICS IN MEDICINE AND BIOLOGY 49 (2004) 1329 1338, a plurality of types of acoustic detectors having different sensitivity characteristics are used to detect acoustic waves at a plurality of positions to reconstruct an image. However, in this case, even when photoacoustic waves actually generated by absorbers have the same sound pressure, an intensity indicated on the reconstructed image may vary according to the size of the absorber.

The present invention has been developed in view of these problems. An object of the present invention is to acquire an appropriate reconstructed image when a plurality of acoustic detectors with different frequency sensitivity characteristics are used.

The present invention provides an apparatus comprising:

a light source;

a plurality of acoustic detectors configured to detect an acoustic wave generated from an object irradiated with light from the light source and output an electric signal, the plurality of acoustic detectors including a first acoustic detector having a first frequency sensitivity characteristics to output a first electric signal and a second acoustic detector having a second frequency sensitivity characteristics, which is different from the first frequency sensitivity characteristics, to output a second electric signal;

a memory configured to hold a correction function created based on a third frequency sensitivity characteristics obtained based on the first and the second frequency sensitivity characteristics; and

an information processor configured to: (a) read the correction function from the memory, (b) correct the first and second electric signals in accordance with the correction function, thereby acquiring corrected first and second electric signals, and, (c) generate image data representing object information using the corrected first and second electric signals.

The present invention also provides an apparatus comprising:

a light source;

a plurality of acoustic detectors configured to detect an acoustic wave generated by an object irradiated with light from the light source and output an electric signal, the plurality of acoustic detectors including a first acoustic detector having a first frequency sensitivity characteristics to output a first electric signal and a second acoustic detector having a second frequency sensitivity characteristics, which is different from the first frequency sensitivity characteristics, to output a second electric signal;

a memory configured to hold a correction image filter created based on a third frequency sensitivity characteristics obtained based on the first and the second frequency sensitivity characteristics; and

an information processor configured to: (a) read the correction image filter from the memory, (b) correct image data based on the first electric signal and image data based on the second electric signal in accordance with the correction image filter, and, (c) generate image data representing object information.

The present invention allows acquisition of an appropriate reconstructed image when a plurality of acoustic detectors with different frequency sensitivity characteristics are used.

Further features of the present invention will become apparent from the following description of exemplary embodiments with reference to the attached drawings.

### BRIEF DESCRIPTION OF THE DRAWINGS

FIGS. 1A and 1B are diagrams illustrating a relation between the frequency and sensitivity of acoustic detectors;

FIG. 2 is a schematic diagram illustrating an apparatus configuration;

FIGS. 3A to 3C are diagrams illustrating frequency sensitivity characteristics of the acoustic detectors;

FIG. 4 is a diagram illustrating a correction function;

FIGS. 5A and 5B are diagrams illustrating results of correction of the frequency sensitivities of the acoustic detectors;

FIGS. 6A to 6F are diagrams illustrating correction of the frequency sensitivity characteristics;

FIG. 7 is a flowchart illustrating the contents of a process;

FIG. 8 is a schematic diagram illustrating another apparatus configuration;

FIG. 9 is a flowchart illustrating the contents of another process; and

FIGS. 10A to 10D are diagrams depicting examples of images acquired.

### DESCRIPTION OF THE EMBODIMENTS

A preferred embodiment of the present invention will be described below with reference to the drawings. Dimensions, materials, shapes, and relative arrangements of components described below should be changed as needed according to a configuration of an apparatus to which the present invention is applied and various conditions for the apparatus. Therefore, the dimensions, materials, shapes, and relative arrangements of components described below are not intended to limit the scope of the present invention to the following description.

The present invention relates to a technique for detecting an acoustic wave propagating from an object to generate object information that is characteristics information on the interior of the object. Therefore, the present invention is considered to be an object information acquiring apparatus and a control method therefor, or an object information acquiring method and a signal processing method. The present invention is also considered to be a program allowing the methods to be executed by an information processing apparatus including hardware resources such as a CPU and memory, and a storage medium that stores the program.

The object information acquiring apparatus in the present invention includes an apparatus that receives an acoustic wave generated, due to a photoacoustic effect, inside an object irradiated with light (electromagnetic wave) to acquire object information in the form of image data. Such an apparatus may also be referred to as a photoacoustic apparatus, a photoacoustic tomography apparatus, a photoacoustic imaging apparatus, or the like. The object information is characteristic value information that is generated using a reception signal resulting from reception of a photoacoustic wave and that corresponds to each of a plurality of positions in the object.

The object information is a value reflecting the absorptivity of optical energy. For example, the object information includes a source of an acoustic wave resulting from light irradiation, an initial sound pressure in the object or an optical-energy absorption density and an absorption coefficient derived from the initial sound pressure, or the concentration of a substance forming a tissue. The object information may be a relative value calculated based on the above-described values. For substance concentrations, an oxyhemoglobin concentration and a deoxyhemoglobin concentration are determined to allow an oxygen saturation distribution to be calculated. Furthermore, a glucose concentration, a collagen concentration, a melanin concentration, or the volume fraction of fat or water may be determined. In addition, two- or three-dimensional object information distribution may be obtained based on object information on different positions in the object. Distribution data may be generated in the form of image data.

The acoustic wave as used herein is typically an ultrasonic wave and includes an elastic wave referred to as a sound wave or an acoustic wave. An electric signal into which an acoustic wave is converted by a transducer or the like is also referred to as an acoustic signal. The ultrasonic wave or acoustic wave as described herein is not intended to limit the wavelengths of these elastic waves. An acoustic wave generated due to the photoacoustic effect is referred to as a photoacoustic wave. An electric signal derived from a photoacoustic wave is also referred to as a photoacoustic signal.

The object information acquiring apparatus can use, as a measurement target, a living organism such as a human being or an animal, a sample other than the living organisms, or a calibration sample such as a phantom. When the object is a living organism, the object information acquiring apparatus can be utilized for diagnosis of blood vessel diseases, follow-up of chemical treatment, and the like.

(Frequency Sensitivity Characteristic)

Now, sensitivity characteristics (frequency sensitivity characteristics) for the frequency band of an acoustic detector will be further discussed. The sensitivity of the acoustic detector decreases gradually as the frequency deviates from the most sensitive frequency. Therefore, the sensitivity is not uniform even in a sensitive frequency band. As described above, the dominant frequency of a photoacoustic signal varies according to the size of an absorber. Thus, when the sensitivity of the acoustic detector varies according to the frequency, the accuracy of reproduction of a generated acoustic wave decreases. As a result, the intensity indicated in a reconstructed image depends on the size of the absorber.

A method for making the sensitivity uniform regardless of the frequency is to recover the components of a frequency band with a reduced sensitivity using a signal deconvolution process. However, when a plurality of acoustic detectors sensitive indifferent frequency bands are used, it is difficult to make the sensitivity uniform simply by executing the deconvolution process.

### Embodiment 1

In the present invention, signals from a plurality of acoustic detectors sensitive indifferent frequency bands are corrected with the frequency bands taken into account. This reduces the dependence of the reconstructed image on the size of the absorber. The principle of the invention, components, an implementation method, and a correction method will be described below in this order.

(Principle)

The dependence of the sensitivity of the acoustic detector on the frequency is the cause of the dependence of the intensity in the reconstructed image on the size of the absorber. Thus, reconstruction can be appropriately achieved by making the sensitivity of the acoustic detector uniform regardless of the frequency. However, when a plurality of acoustic detectors sensitive in different frequency bands are used, the signals from the acoustic detectors are added together during the reconstruction. Consequently, even when the sensitivity of each of the acoustic detectors is made uniform at different frequencies, the sensitivity may fail to be uniform in an image resulting from the addition of the signals.

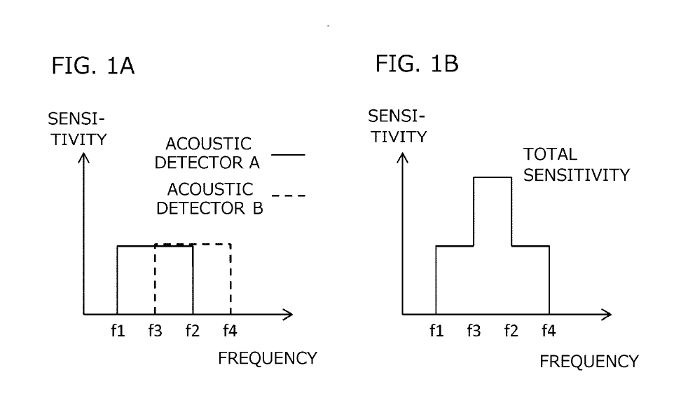

Further description will be given with reference to FIGS. 1A and 1B. The axis of abscissas represents frequency, and the axis of ordinate represents sensitivity. In FIG. 1A, a solid line indicates the sensitivity characteristics of an acoustic detector A that is sensitive in a frequency band from frequency f1 to frequency f2. A dashed line indicates the sensitivity characteristics of an acoustic detector B that is sensitive in a frequency band from frequency f3 to frequency f4. The acoustic detectors A and B exhibit an equal sensitivity in the sensitive frequency band. If f3<f2, the frequency components from frequency f3 to frequency f2 are detected by both acoustic detectors, and the frequency components from frequency f1 to frequency f3 are detected only by one of the acoustic detectors, whereas the frequency components from frequency f2 to frequency f4 are detected only by the other acoustic detector. This is equivalent to the fact that the frequency components from frequency f3 to frequency f2 are detected with a sensitivity double the sensitivity with which the frequency components from frequency f1 to frequency f3 are detected and with which the frequency components from frequency f2 to frequency f4 are detected.

The above description of course holds true given that the acoustic detectors A and B are located in spatially the same area. The above description also holds true even when the acoustic detectors A and B are located in different areas. This is because the addition of the signals from the acoustic detectors A and B is performed by reconstruction. Therefore, the total sensitivity resulting from addition of the sensitivities of the acoustic detectors A and B can be used to consider the frequency sensitivity in the space (that is, the reconstructed image) in which the acoustic detectors A and B contribute to reconstruction.

In FIGS. 1A and 1B, in the reconstructed image, an absorber sized to make the frequency components from frequency f3 to frequency f2 dominant appears with high intensity. In the present invention, with the overlapping of the sensitivities of the plurality of acoustic detectors taken into account, the sensitivity is made uniform so as to avoid depending on the frequency. Specifically, the signals from the acoustic detectors are corrected so as to make the total sensitivities of the acoustic detectors uniform.

(Components)

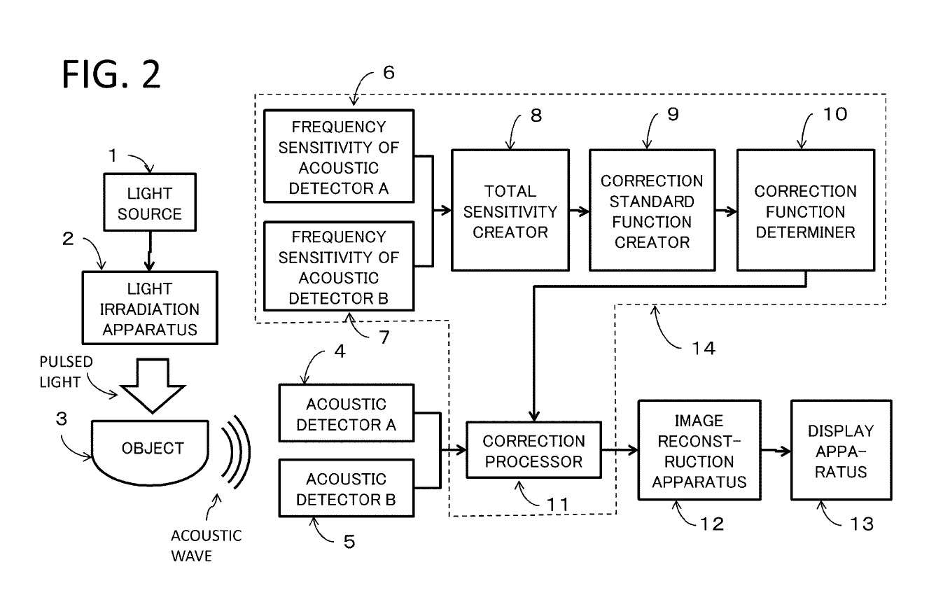

Components of the present invention will be described using FIG. 2. The components of the present invention are a light source 1, a light irradiation apparatus 2, the acoustic detector A (4), an acoustic detector B (5), a signal processing apparatus 14, an image reconstruction apparatus 12, and a display apparatus 13. A measurement target is an object 3. The signal processing apparatus 14 includes a total-sensitivity creator 8, a correction standard function creator 9, a correction function determiner 10, and a correction processor 11. A frequency sensitivity 6 of the acoustic detector A and a frequency sensitivity 7 of the acoustic detector B are saved to a memory or the like not depicted in the drawings. The signal processing apparatus includes a memory in which a correction function and a filter generated using a method in the present invention are held. Alternatively, the signal processing apparatus communicates with a storage apparatus serving as a memory to transmit and receive information to and from the storage apparatus. Timings when the correction function and the filter are generated are optional and may be pre-defined and saved to the memory or may be the same as timings when acoustic signals are processed.

(Light Source)

The light source 1 is an apparatus that generates pulsed light. The light source 1 is desirably a laser in order to provide high power but may be a light emitting diode or the like. To allow a photoacoustic wave to be effectively generated, the object may be irradiated with light in a sufficiently short time according to thermal characteristics of the object. When the object is a living organism, pulsed light emitted from the light source desirably has a pulse width of several tens of nanoseconds or less. The pulsed light desirably has a pulse width that makes the pulsed light likely to reach the absorber. When hemoglobin in a living organism is measured, the pulse width is desirably approximately 700 nm to 1200 nm in a near infrared region referred to as a biological window. Light in this region reaches a relatively deep portion of the living organism and thus enables information on the deep portion to be acquired. The wavelength of the pulsed light desirably exhibits a high absorption coefficient for an observation target. To obtain spectral information such as oxygen saturation, a plurality of light sources with different wavelengths need to be used to provide respective photoacoustic signals. In this case, to reduce propagation of computational errors, wavelengths leading to significantly different absorption coefficients of spectral components are desirably used.

(Light Irradiation Apparatus)

The light irradiation apparatus 2 guides pulsed light generated by the light source 1 to the object 3. Specifically, the light irradiation apparatus 2 is optical equipment such as an optical fiber, a lens, a mirror, or a diffuser. To acquire a wide range of data, the light irradiation apparatus 2 may be allowed to scan irradiation positions of the pulsed light. In this case, the scan may be performed in conjunction with operation of the acoustic detectors (4 and 5). For increased signal acquisition efficiency, an area irradiated with light desirably coincides with a range within which the acoustic detectors are sensitive. To obtain spectral information, photoacoustic signals with different wavelengths need to be acquired. Consequently, the object is irradiated with pulsed light with different wavelengths at the respective timings. The optical equipment is not limited to the above-described equipment. Any type of equipment may be used so long as the equipment achieves the above-described functions.

(Object)

The object 3 is a measurement target. The object 3 may be a living organism or a phantom that simulates acoustic characteristics and optical characteristics of the living organism. The photoacoustic apparatus enables imaging of an absorber that is present inside the object and that has a large light absorption coefficient. For the living organism, examples of the absorber include hemoglobin, water, melanin, collagen, and fat. For the phantom, a substance that simulates the optical characteristics of the above-described living organism is sealed in the object as an absorber. The living organism involves individual differences in shape and characteristics. An alternative object may be a living organism or a phantom into which a contrast dye or a molecular probe is injected.

(Acoustic Detectors)

The acoustic detector A (4) and the acoustic detector B (5) include respective elements that convert an acoustic wave into an electric signal. The acoustic detector A (4) and the acoustic detector B (5) have different frequency sensitivities. The frequency sensitivity refers to sensitivity characteristics at each frequency as illustrated in FIGS. 1A and 1B. In actuality, even the same type of elements vary in frequency sensitivity due to limits in a manufacturing process. However, in the following description, a variation among the same type of elements with respect to a design value is assumed to fall within a tolerable range based on measurement accuracy. On the other hand, the acoustic detector A (4) and the acoustic detector B (5) originally have different designed frequency sensitivities.

Desirably, the acoustic detector A (4) and the acoustic detector B (5) are sensitive in different frequency bands, and both acoustic detectors are sensitive in a part of the frequency bands. In other words, the sensitive frequency bands of the two acoustic detectors preferably have an overlapping portion. This state is simply illustrated in FIGS. 1A and 1B. “Being sensitive” as used herein means that the sensitivity is higher than a certain threshold. The threshold is determined based on a noise level, a user's specification, a comparison with a frequency corresponding to the peak of the sensitivity, or the like. For example, when a peak frequency is set as a frequency in which the sensitivity exhibits the peak, a frequency in which the sensitivity is equal to or more than 50% of the sensitivity in the peak frequency may be a sensitive frequency. However, the present invention is applicable even when the sensitive frequency bands of the two acoustic detectors fail to overlap or when both acoustic detectors have the same sensitive frequency band but differ from each other in sensitivity at each frequency. An element size makes an effect on the sensitivity. However, the sizes of the acoustic detector A (4) and the acoustic detector B (5) are desirably varied so as to have substantially the same directionality.

Partial overlapping of the sensitive frequency bands of the acoustic detector A (4) and the acoustic detector B (5) allows continuous widening of the frequency band in which the apparatus as a whole is sensitive. As a result, the present invention is effective on all absorbers with sizes corresponding to the lower limit frequency to the upper limit frequency for the total sensitivity. Three or more types of acoustic detectors may be used.

For accurate reconstruction, an acoustic wave is desirably detected in as many areas as possible. This allows virtual images from being formed. For uniform image quality, each of the acoustic detector A (4) and the acoustic detector B (5) is more desirably installed in a plurality of areas. Members each of which is a combination of the acoustic detector A (4) and the acoustic detector B (5) may be installed in a plurality of areas. Acoustic waves may be detected in a plurality of areas by moving the acoustic detector A (4) and the acoustic detector B (5) using a scanning mechanism such as an XY stage.

For uniform image quality, the acoustic detectors A (4) and the acoustic detectors B (5) desirably avoid being installed in particular areas in a concentrative manner and are uniformly mixed together. Specifically, the acoustic detectors A (4) and the acoustic detectors B (5) may be alternately installed on a certain surface. Typically, the acoustic detectors have directionality, and thus, the sensitivity of the acoustic detectors decreases with increasing distance from the front. Thus, each acoustic detector is desirably installed such that a direction in which the acoustic detector is sensitive coincides with a space intended for reconstruction (reconstruction space). This enables each acoustic detector to observe the reconstruction space, allowing image quality to be improved and made uniform. To meet these conditions, the acoustic detector A (4) and the acoustic detector B (5) may be installed on a spherical surface, and the front of the acoustic detectors may be directed toward the center of the sphere. Such an installation method can be executed by arranging each acoustic detector in a semispherical or spherical-crown-like supporter.

In the photoacoustic apparatus, acoustic waves generated inside the object 3 are received by the acoustic detector A (4) and the acoustic detector B (5). Thus, to prevent acoustic waves from being reflected or attenuated, the acoustic detector A (4) and the acoustic detector B (5) need to be installed so as to be acoustically coupled to the object 3. An acoustic matching material such as an acoustic matching gel, water, or oil is desirably provided between each acoustic detector and the object 3. The acoustic detectors desirably have high sensitivity and wide frequency bands. Specifically, the acoustic detectors include PZTs, PVDF, cMUTs, or Fabry-Perot interferometers. However, the acoustic detectors are not limited to these examples. Any acoustic detectors may be used so long as the acoustic detectors fulfill the appropriate functions.

(Signal Processing Apparatus)

The signal processing apparatus 14 processes signals provided by the acoustic detectors. Signal processing is desirably digital signal processing that enables flexible processing. Thus, before the processing is executed, analog signals output from the acoustic detectors are converted into digital signals by an analog-digital converter (ADC) not depicted in the drawings. For analog signal processing, a digital conversion is performed on an output from the signal processing apparatus 14. Specifically, the signal processing apparatus 14 is, for the digital signal processing, for example, a field-programmable gate array (FPGA) or a computer that operates utilizing arithmetic resources such as a CPU and memory in accordance with a program. The signal processing apparatus 14 is an electric circuit for the analog signal processing. Units included in the signal processing apparatus 14 and the contents of the processing will be described below. The signal processing apparatus includes a memory apparatus serving as a memory. Alternatively, an external storage apparatus may be utilized as the memory.

(Image Reconstruction Apparatus)

The image reconstruction apparatus 12 reconstructs a digital signal to create an initial sound pressure distribution. For reconstruction, any of known approaches can be adopted such as back projection, phasing addition, Fourier transform, a model-based method, and time reversal. During image reconstruction, an output from the signal processing apparatus 14 is propagated backward from the positions of the acoustic detector A (4) and the acoustic detector B (5) to create one image. An absorption coefficient distribution may be created based on the distribution of the amount of light in the object and the initial sound pressure distribution. Component information such as the oxygen saturation distribution may be acquired based on the results of measurement using light with a plurality of wavelengths. The image reconstruction apparatus 12 may also include an FPGA, a computer, or an electric circuit. The signal processing apparatus and the image reconstruction apparatus correspond to an information processor in the present invention. The information processor is a unit that generates image data indicative of object information based on signals output from the acoustic detectors. The functions executed by the information processor may be implemented by different pieces of hardware.

(Display Apparatus)

The display apparatus 13 displays images provided by the image reconstruction apparatus 12. The display apparatus 13 may be a liquid crystal display, a plasma display, or the like. The display apparatus 13 may be included in a part of the photoacoustic apparatus in the present invention or provided separately from the photoacoustic apparatus.

Now, the units included in the signal processing apparatus 14 will be described.

(Frequency Sensitivities of the Acoustic Detectors)

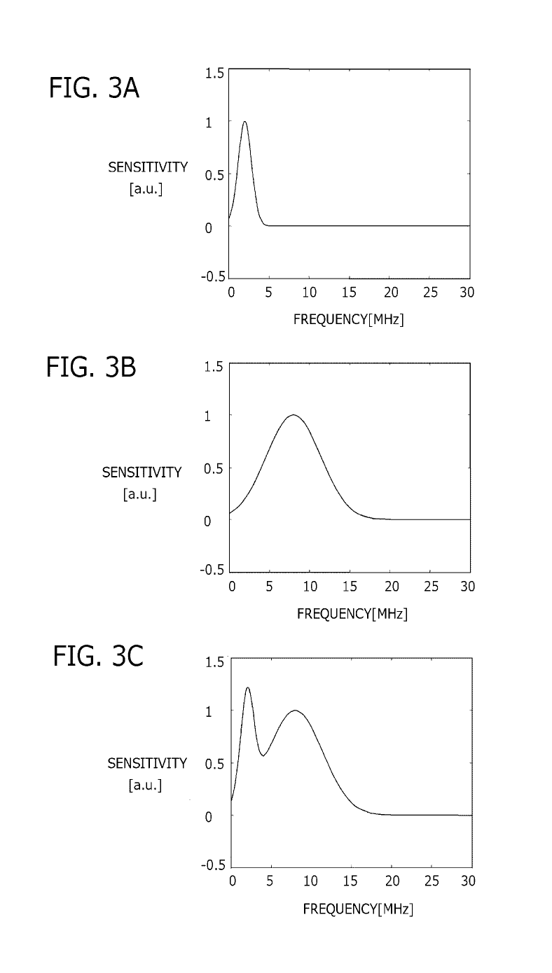

FIG. 3A illustrates an example frequency sensitivity 6 of the acoustic detector A, and FIG. 3B illustrates an example frequency sensitivity 7 of the acoustic detector B. These frequency sensitivities are used to create a correction function. The sensitivities are acquired based on pre-measurement values, design information values, values obtained by executing optimization calculation such as blind deconvolution on signals provided by the acoustic detectors, or the like, and are stored in the memory. When a plurality of the acoustic detectors A (4) and a plurality of the acoustic detectors B (5) are provided, the acoustic detectors may be provided with different frequency sensitivities. However, a common value is preferably used in order to avoid complication of the processing.

(Total-Sensitivity Creator)

The total-sensitivity creator 8 executes a process of adding the frequency sensitivity 6 of the acoustic detector A and the frequency sensitivity 7 of the acoustic detector B together. FIG. 3C illustrates an example result of the addition. The addition is a process represented by Expression (1).

S_{SUM}(f)=S_{A}(f)×D_{A}+S_{B}(f)×D_{B} (1)

A total sensitivity (third frequency sensitivity characteristic) that is a function of a frequency f is denoted as S_{SUM}(f). The frequency sensitivity of the acoustic detector A (first frequency sensitivity characteristics) is denoted as S_{A}(f). The frequency sensitivity of the acoustic detector B (second frequency sensitivity characteristics) is denoted as S_{B}(f). The directionality-based sensitivity of the acoustic detector A contributing to the reconstruction space is denoted as D_{A}. The directionality-based sensitivity of the acoustic detector B contributing to the reconstruction space is denoted as D_{B}. When a plurality of the acoustic detectors A and a plurality of the acoustic detectors B are provided, the sensitivity based on directionality varies according to area. Thus, the product of each frequency sensitivity and the corresponding directionality-based sensitivity is added to Expression (1). The directionality may be simply set based on a normal direction of an acoustic-wave reception surface.

Precise reconstruction needs execution, for each voxel, of processing based on an angle to the acoustic detector. However, this processing is complicated and involves heavy arithmetic loads. Thus, the acoustic detectors may be installed somewhat away from the reconstruction space to make the directionality-based sensitivities of the acoustic detectors equivalent to one another. In this case, the total sensitivity can be calculated in accordance with Expression (2).

S_{SUM}(f)=S_{A}(f)+S_{B}(f) (2)

When a plurality of the acoustic detectors A and a plurality of the acoustic detectors B are provided, the total sensitivity is calculated in accordance with Expression (3). In Expression (3), the number of the acoustic detectors A is denoted as N_{A}, and the number of the acoustic detectors B is denoted as N_{B}.

S_{SUM}(f)=S_{A}(f)×N_{A}+S_{B}(f)×N_{B} (3)

In a simplified method, the total sensitivity may be replaced with an average sensitivity resulting from averaging of the frequency sensitivity 6 of the acoustic detector A and the frequency sensitivity 7 of the acoustic detector B. Expressions (4) to (6) represent calculation methods for the average sensitivity corresponding to Expressions (1) to (3). In Expressions (4) to (6), the average sensitivity, expressed as a function of a frequency f, is denoted as Sₘₑₐₙ(f).

$$\begin{matrix}[ Math . 1 ] & \\ {S}_{mean} ( f ) = \frac{{S}_{A} ( f ) \times {D}_{A} + {S}_{B} ( f ) \times {D}_{B}}{2} & ( 4 ) \\ {S}_{mean} ( f ) = \frac{{S}_{A} ( f ) + {S}_{B} ( f )}{2} & ( 5 ) \\ {S}_{mean} ( f ) = \frac{{S}_{A} ( f ) \times {N}_{A} + {S}_{B} ( f ) \times {N}_{B}}{{N}_{A} + {N}_{B}} & ( 6 )\end{matrix}$$

(Correction Standard Function Creator)

The correction function determiner 10 described below determines the correction function so as to make the total sensitivity flat. The correction standard function creator 9 determines a frequency sensitivity function that makes the total sensitivity flat and that corresponds to a correction target. The correction standard function is desirably such that the sensitive frequency band corresponding to the total sensitivity is flat like a table top. However, the sensitive frequency band need not be shaped like a perfect table top and may have any such a shape so long as the sensitivity of the correction standard function is flattened compared to the uncorrected total sensitivity. The flattening in the present invention refers to a reduced variation in sensitivity, that is, the sensitivity having a reduced standard deviation and a reduced variance. “Being sensitive at a certain frequency” means that the sensitivity at that frequency is higher than a predetermined threshold (first threshold) determined based on the noise level or the like of the acoustic detector. A method for determining the threshold is to measure only noise and to determine the threshold to be the average value for the frequency spectrum of the noise. To avoid emphasizing the noise, the correction standard function desirably changes the total sensitivity as insignificantly as possible. The correction standard function is saved to the memory. Alternatively, immediately after the correction standard function is created, the subsequent process may be executed.

Methods (I) to (VI) in which the correction standard function creator 9 generates such a correction standard function that satisfies the above-described conditions are explained.

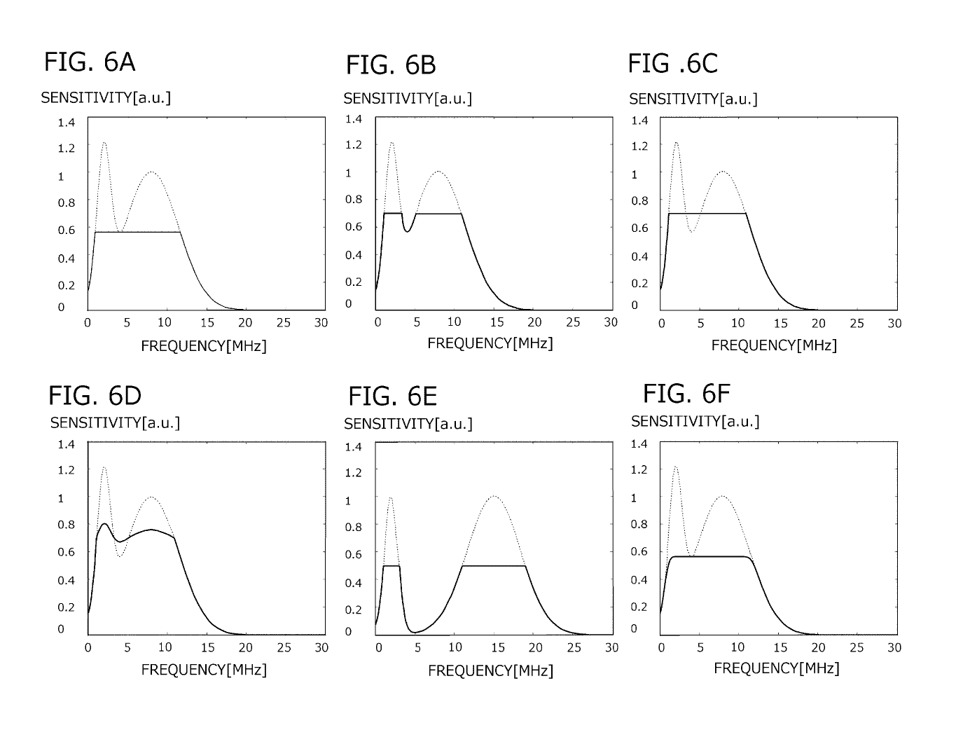

(I) The correction standard function creator 9 calculates the upper limit frequency and the lower limit frequency of the sensitive frequency band corresponding to the total sensitivity, and detects a valley-like portion of the sensitivity within the corresponding range using differential values or the like. Then, the values of the sensitivity larger than the value at the bottom of the valley are reduced to the value at the bottom of the valley. The correction standard function represented by a solid line in FIG. 6A has been generated based on the total sensitivity represented by a dashed line in accordance with this method.

(II) The correction standard function creator 9 makes, in the total sensitivity within the sensitive frequency band, values larger than a certain threshold (a second threshold larger than the first threshold) the same as the second threshold. A correction standard function resulting from this method is illustrated in FIG. 6B.

(III) In addition to executing the method (II), the correction standard function creator 9 makes all the values of the total sensitivity within the sensitive frequency band the same as the second threshold. A correction standard function resulting from this method is illustrated in FIG. 6C.

(IV) The correction standard function creator 9 decreases, in the total sensitivity within the sensitive frequency band, the intensity of the total sensitivity with respect to the threshold at a constant rate so as to reduce a variation in the total sensitivity. A correction standard function resulting from this method is illustrated in FIG. 6D.

(V) When the sensitive frequency bands of the acoustic detector A (4) and the acoustic detector B (5) do not overlap, a possible method is to make the values of frequencies with a total sensitivity higher than a certain threshold equal to the value corresponding to the threshold. The threshold may be a threshold that allows the presence or absence of the sensitivity to be determined or another value larger than the value corresponding to the threshold. A correction standard function resulting from this method is illustrated in FIG. 6E.

(VI) For each of the above-described correction standard functions, the opposite ends of the flat top are shaped like cliffs, which may distort the signal. Thus, the correction standard function may be convoluted with a Gaussian distribution or the like so as to be smoothed. As an example, FIG. 6F is provided that illustrates a correction standard function resulting from convolution of the correction standard function in FIG. 6A with the Gaussian distribution. The above-described process for obtaining the correction standard function may be manually executed by a manipulator or automatically in accordance with a rule.

(Correction Function Determiner)

The correction function determiner 10 determines a correction function for the acoustic detector A and the acoustic detector B so as to make the total sensitivity equal to the correction standard function. When the correction standard function is prevented from exceeding the total sensitivity in all of the frequency band, the correction function can be created in accordance with Expression (7). In Expression (7), a correction function common to the acoustic detector A and the acoustic detector B is denoted as C_{A,B}(f), and the correction standard function is denoted as Sₜ(f). The correction function determiner 10 acquires the correction standard function from the memory or the correction standard function creator to create a correction function and allows the correction function to be held in the memory. Alternatively, immediately after the correction function is created, the subsequent process may be executed.

$$\begin{matrix}[ Math . 2 ] & \\ {C}_{A , B} ( f ) = \frac{St ( f )}{{S}_{sum} ( f )} & ( 7 )\end{matrix}$$

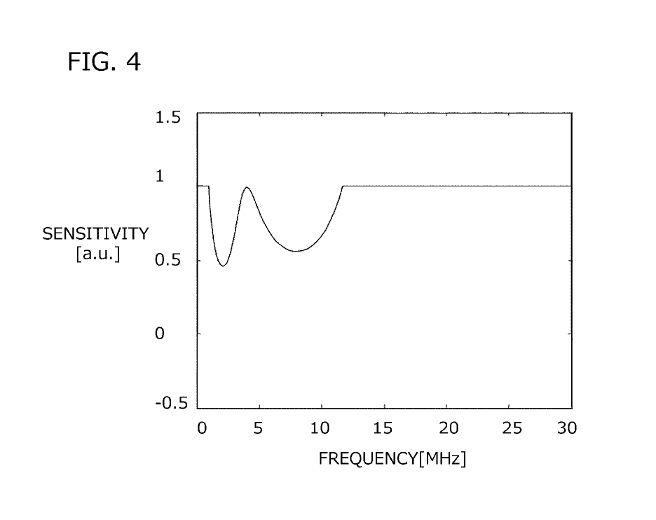

A correction function calculated by this method is illustrated in FIG. 4. This correction function is common to the acoustic detector A and the acoustic detector B. This allows the processing to be simplified even when a large number of acoustic detectors with different frequency sensitivities are provided. Separate correction functions may be used so long as correction functions C_{A}(f) and C_{B}(f) for the acoustic detector A and the acoustic detector B are determined so as to satisfy Expression (7). For example, the correction functions may be determined by a method using Expressions (8) and (9) described below.

S_{A}(f)×C_{A}(f)+S_{B}(f)×C_{B}(f)=Sₜ(f) (8)

In FIGS. 6C and 6D, the sensitivity of the correction standard function is higher than the total sensitivity in a certain portion of the frequency band (corresponding to the bottom of the valley of the total sensitivity). In this case, C_{A}(f) and C_{B}(f) are desirably determined, with bottom valleys S_{A}(f) and S_{B}(f) taken into account. This is because, at the bottom of the valley, the signal is emphasized, so that, when C_{A}(f) and C_{B}(f) are determined with S_{A}(f) and S_{B}(f) not taken into account, noise is emphasized if one of the sensitivities S_{A}(f) and S_{B}(f) is low. Thus, the frequency sensitivity of the acoustic detector may be considered to be a weight for the correction function so that the correction function may be increased in a portion of the frequency band where the acoustic detector is very sensitive. That is, Expression (9) is taken into account, and C_{A}(f) and C_{B}(f) are determined by solving simultaneous equations represented by Expressions (8) and (9).

$$\begin{matrix}[ Math . 3 ] & \\ \frac{{S}_{A} ( f )}{{C}_{A} ( f )} = \frac{{S}_{B} ( f )}{{C}_{B} ( f )} & ( 9 )\end{matrix}$$

The frequency components of each acoustic detector other than the sensitive frequency band are noise components. Thus, the correction function for each acoustic detector desirably makes the values in the frequency band zero. This improves the SN ratio of the reconstructed image. In this case, to avoid degradation of image quality resulting from distortion of the signal, transition sections are desirably made smooth.

When three types of acoustic detectors with different frequency sensitivities are provided, the correction function can be calculated based on simultaneous equations using Expressions (10) and (11). The frequency sensitivity of a third acoustic detector C is denoted as S_{C}(f), and a correction function for the acoustic detector C is denoted as C_{C}(f). This also applies to four or more types of acoustic detectors.

$$\begin{matrix}[ Math . 4 ] & \\ {S}_{A} ( f ) \times {C}_{A} ( f ) + {S}_{B} ( f ) \times {C}_{B} ( f ) + {S}_{C} ( f ) \times {C}_{C} ( f ) = St ( f ) & ( 10 ) \\ \frac{{S}_{A} ( f )}{{C}_{A} ( f )} = \frac{{S}_{B} ( f )}{{C}_{B} ( f )} = \frac{{S}_{C} ( f )}{{C}_{C} ( f )} & ( 11 )\end{matrix}$$

(Correction Processor)

The correction processor 11 corrects each of the acoustic signals from the acoustic detector A (4) and the acoustic detector B (5) using the correction function obtained by the correction function determiner 10. Specifically, as represented by Expression (12), the correction processor 11 converts the resultant signal into a frequency domain signal, multiplying the signal by the correction function, and executing a process of returning the signal to a time domain signal using a frequency filter. The correction processor acquires the correction function from the memory or the correction function determiner. An uncorrected signal output from the acoustic detector A (4) is denoted as Sig_{A}(t). A corrected signal resulting from the correction process is denoted as Sig′_{A}(t). A Fourier operator is denoted as F, and an inverse Fourier operator is denoted as F⁻¹.

Sig′_{A}(t)=F⁻¹(F(Sig_{A}(t))×C_{A}W) (12)

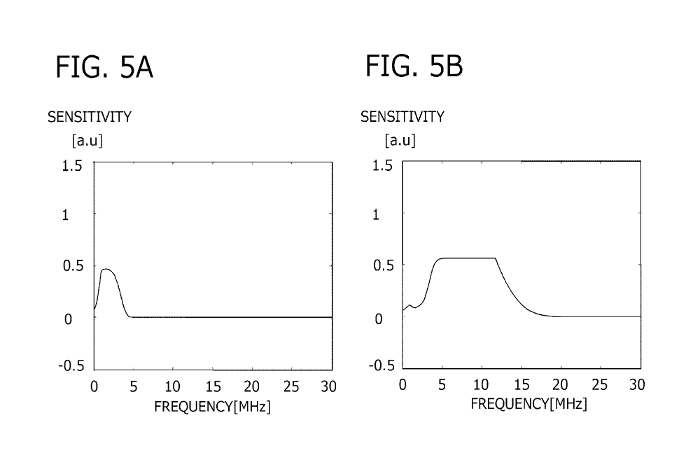

The correction processor 11 may execute the correction process in the time domain. That is, the correction function is converted into a time domain function, and the resultant function is convoluted with the signal in the time domain. Given that the correction function is common to the acoustic detector A (4) and the acoustic detector B (5), the same correction function is applied to the acoustic signals from the acoustic detector A (4) and the acoustic detector B (5). Given that different correction functions are used for the respective acoustic detectors, the correction functions corresponding to the respective acoustic signals are applied. FIGS. 5A and 5B illustrate the results of multiplication of the frequency sensitivities of the acoustic detector A (4) and the acoustic detector B (5), respectively, by the correction function.

(Process Flow)

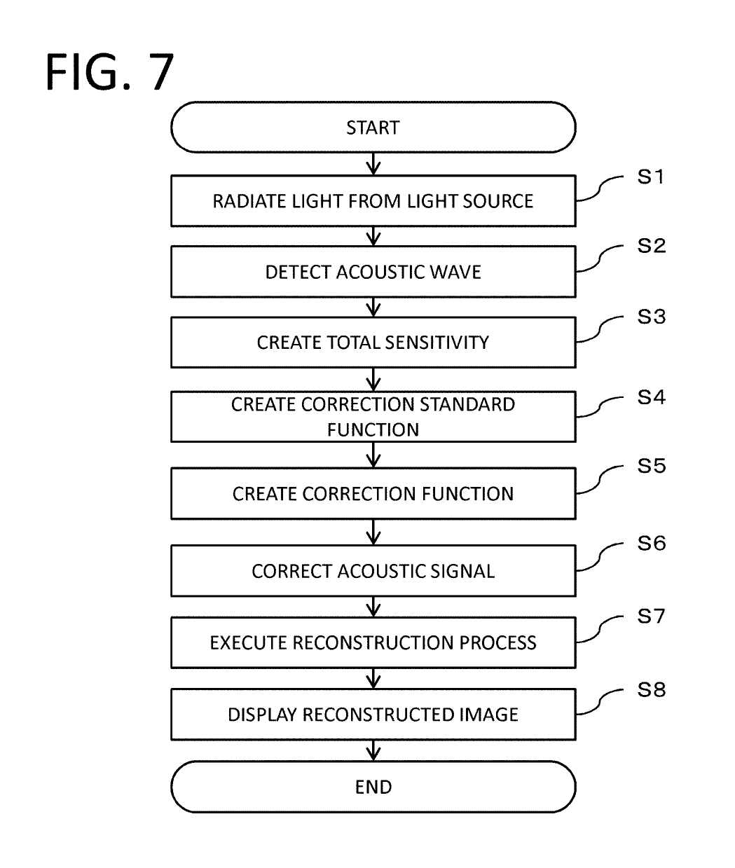

Now, a process method in the present embodiment will be described using FIG. 7. First, the light source 1 irradiates the object with pulsed light (step S1). Subsequently, the acoustic detectors receive acoustic waves generated by the object (step S2). The total-sensitivity creator 8 then creates the total sensitivity based on the frequency sensitivities of the acoustic detectors (step S3). The correction standard function creator 9 subsequently creates a correction standard function (step S4). The correction function determiner 10 then creates a correction function (step S5). The acquisition of the correction function may be achieved by reading a correction function pre-saved in the memory and corresponding to the measurement conditions.

Then, the correction processor 11 corrects the acoustic signals using the correction function (step S6). The image reconstruction apparatus 12 then performs reconstruction using the corrected signals (step S7). Finally, the display apparatus 13 displays the reconstructed image (step S8). The operations in S3 to S5 may be performed before the start of the process. When the frequency sensitivities of the acoustic detectors are obtained by blind deconvolution of the acoustic signals or the noise level is estimated based on the acoustic signals, preliminary measurement may be performed before the start of the measurement or the signal obtained in S2 may be used.

The photoacoustic apparatus in the present embodiment reduces the dependence of the image intensity on the size of the absorber, providing a reconstructed image that facilitates quantitative evaluation.

### Embodiment 2

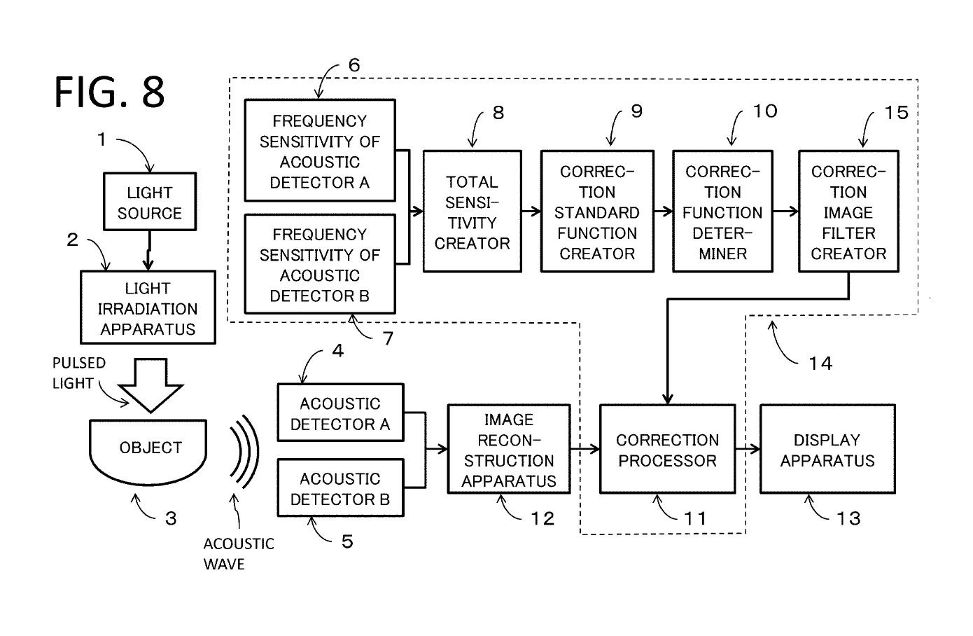

In Embodiment 1, signals are corrected. In the present embodiment, the effects of the invention are produced by executing a correction process on a reconstructed image. Components of the present embodiment will be described using FIG. 8. Compared to Embodiment 1, the present embodiment additionally includes a correction image filter creator 15 as a unit included in the signal processing apparatus 14. Furthermore, the arrangement order of the image reconstruction apparatus 12 and the correction processor 11 is reversed, and the image reconstruction apparatus 12 and the correction processor 11 in the present embodiment are different, in the contents of processing, from the image reconstruction apparatus 12 and the correction processor 11 in Embodiment 1. The other components are the same as the corresponding components in Embodiment 1, and description thereof is simplified.

(Image Reconstruction Apparatus)

The image reconstruction apparatus 12 executes a reconstruction process similar to the reconstruction process in Embodiment 1 on signals output from the acoustic detector A (4) and the acoustic detector B (5) to obtain an image A and an image B. When a plurality of the acoustic detectors A (4) and a plurality of acoustic detectors B (5) are provided, the image A may be generated based on signals from the group of the acoustic detectors A, and the image B may be generated based on signals from the group of the acoustic detectors B. After generating an initial sound pressure distribution, the image reconstruction apparatus 12 in the present embodiment outputs the initial sound pressure distribution to the succeeding unit without generating an absorption coefficient distribution or the like.

(Correction Image Filter Creator)

The correction image filter creator 15 converts correction functions for the signals from the acoustic detector A (4) and the acoustic detector B (5) provided by the correction function determiner 10, into reconstructed image filters for the image A and the image B. Like the other components, the correction image filter creator 15 may be implemented using a circuit or an information processing circuit.

Conversion of a correction function into a correction image filter is performed by projecting the correction function on a two- or three-dimensional space in the form of a concentric circle. In an orthogonal coordinate system, the correction image filter is shaped like a rectangle or a rectangular parallelepiped. Each of the vertices of the rectangle or the rectangular parallelepiped represents a component with the lowest frequency (DC component). The frequency increases consistently with the distance from each vertex. Therefore, the correction image filter creator 15 rotates the correction function for the signal around each vertex and provides a pixel at corresponding coordinates with the intensity of the correction function.

The size of the correction image filter is desirably the same as an image to be corrected. Therefore, the scale of the image filter is determined based on the scale of the reconstructed image. The size (unit number) of a side of the reconstructed image is denoted as r[voxel], the scale is denoted as s[m/voxel], and the propagation velocity of acoustic waves is denoted as v[m/s]. In this case, the maximum frequency fₘₘ[Hz] is as represented by Expression (13), and the frequency per pixel fᵤ[Hz/voxel] is as represented by Expression (14). The voxel refers to a three-dimensional pixel and may be replaced with a pixel when image processing is two-dimensionally executed. Thus, the correction image filter creator 15 converts the correction function into the correction image filter using the scale conversion and the concentrically circular projection.

$$\begin{matrix}[ Math . 5 ] & \\ {f}_{max} = \frac{v}{2 s} & ( 13 ) \\ {f}_{u} = \frac{v}{rs} & ( 14 )\end{matrix}$$

(Correction Processor)

The correction processor 11 adapts, to the images A and the image B provided by the image reconstruction apparatus 12, the corresponding correction image filters provided by the correction image filter creator 15, and superimposes the results on each other. Alternatively, correction image filters may be held in the memory so that the correction processor may acquire desired correction image filters corresponding to the measurement conditions from the memory, as is the case with Embodiment 1.

The created correction image filters are expressed in a spatial frequency domain. Thus, the correction processor 11 in the present embodiment converts the image A and the image B into spatial-frequency-domain images using two- or three-dimensional Fourier transform, multiplies the conversion results by the correction image filters, and subsequently return the images to spatial-domain images. Moreover, the spatial-domain images are preferably added together. When the image A and the image B are denoted as I_{A} and I_{B} and the correction image filters for the respective images are denoted as CI_{A} and CI_{B}, the correction processor produces a result I in accordance with Expression (15). The Fourier operator is denoted as F, and the inverse Fourier operator is denoted as F⁻¹.

I=F⁻¹(F(I_{A})×CI_{A})+F⁻¹(F(I_{B})×CI_{B}) (15)

Thus, a corrected initial sound pressure distribution is obtained. Based on the corrected initial sound pressure distribution and an amount-of-light distribution, an absorption coefficient distribution can be acquired and spectral information distribution and a substance concentration distribution can also be acquired using light with a plurality of wavelengths. The correction processor 11 may execute the correction process in the time domain as is the case with Embodiment 1. In this case, the correction processor 11 converts correction image filters expressed in the spatial frequency domain into time domain filters and applies the resultant filters to the reconstructed images. Possible methods in this case are to use correction image filters suitable for the images A and B, respectively and to use a correction image filter that is created based on output signals from all the acoustic detectors and that is suitable for the images. When object information is acquired by arranging electric signals according to time series, the correction target is not the reconstructed images but object information as described above.

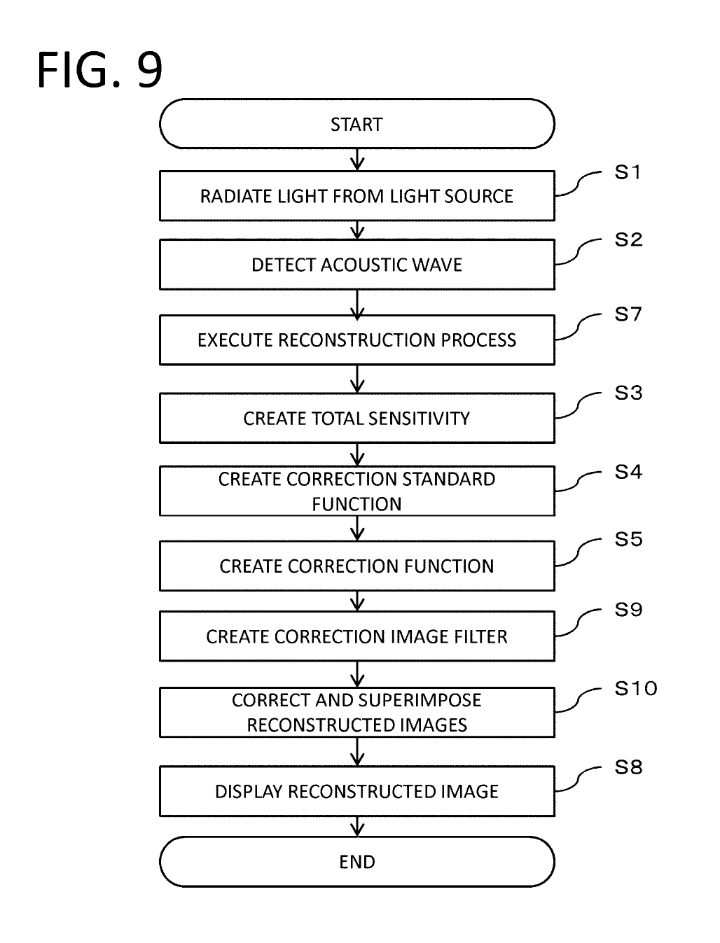

A process method in the present embodiment will be described using FIG. 9. In the present embodiment, after step S2, a reconstruction process is executed using signals from the acoustic detector A (4) and the acoustic detector B (5) (step S7). Subsequently, steps S3 to S5 are executed as is the case with Embodiment 1, and then, the correction image filter creator 15 creates correction image filters (step S9). The correction processor 11 subsequently corrects the reconstructed images using the correction image filters and adds the corrected reconstructed images together (step S10). Finally, the resultant image is displayed (step S8). The operations in steps S3 to S9 may be performed before the start of measurement given that the scale of the reconstructed image and the like are predetermined. The operations in steps S3 to S9 may be executed at anytime before S10.

Using the apparatus in the present embodiment reduces the image intensity on the size of the absorber to provide images that facilitate quantitative evaluation.

### Examples

The effects of the present invention were checked through simulation. As the object, four spherical absorbers are installed on a three-dimensional space. The absorbers were 1.0 mm, 0.5 mm, 0.33 mm, and 0.25 mm, respectively, in diameter. The acoustic detectors were installed on a semispherical surface surrounding the absorbers and having a radius of 100 mm. The spaces among the absorbers and the acoustic detectors were filled with water.

The acoustic detectors used had such sensitivity characteristics as illustrated in FIGS. 3A to 3C. That is, the characteristics of the acoustic detector A were represented by a Gaussian distribution with a full width at half maximum of 2 MHz around 2 MHz. The characteristics of the acoustic detector B were represented by a Gaussian distribution with a full width at half maximum of 8 MHz around 8 MHz. Detection surfaces of the acoustic detectors A and B were shaped like discs with a diameter of 3 mm. The number of the acoustic detectors A was 256, and the number of the acoustic detectors B was 256. A total of 512 acoustic detectors were alternately arranged in an even manner on the semispherical surface. In this configuration, photoacoustic waves generated by the objects were received and digitalized into acoustic signals.

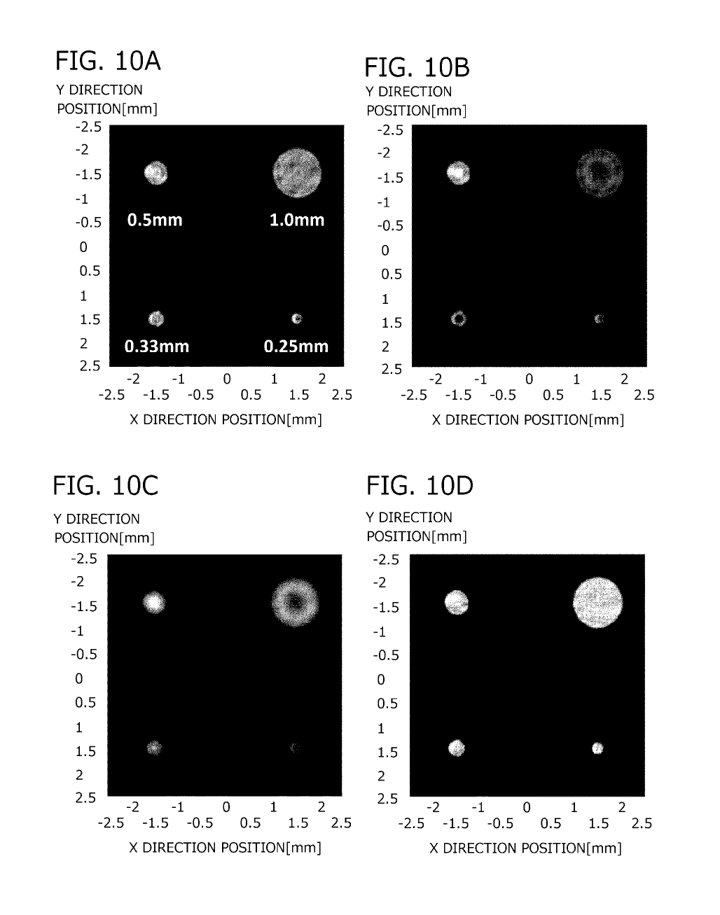

In the present example, as in the method (I) the correction standard function creator 9 created a correction standard function based on a value for a valley portion between peaks for each acoustic detector. The correction function determiner 10 acquired a correction function in accordance with Expression (7). The correction processor 11 corrected the signals using the correction function. An image generated by the image reconstruction apparatus 12 is depicted in FIG. 10A.

Meanwhile, FIG. 10B depicts the result of reconstruction without correction of the signals. FIG. 10C depicts the result of reconstruction through deconvolution on each acoustic detector. FIG. 10D depicts an ideal case where reconstruction was performed using signals involving no dependence of the sensitivity of the acoustic detector on the frequency.

In FIG. 10B, the intensity is high for the 0.5-mm absorber, and blanking is observed for the 0.33-mm absorber. In FIG. 10C, the intensity is high for the 0.5-mm absorber and is low near a central portion of the 1.0-mm absorber. In contrast, in FIG. 10A, which indicates the result of the present invention, the image is slightly dark near the central portion of the 0.25-mm absorber, whereas similar intensities are observed for the other absorbers. In FIG. 10D, which illustrates an ideal case, all the absorbers exhibit the same intensity. This indicates that using the apparatus in the present invention enables a reduction in the dependence of the intensity on the size of the absorber.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-191578, filed on Sep. 29, 2015, which is hereby incorporated by reference herein in its entirety.

What is claimed is:

1. An apparatus comprising:
a light source;
a plurality of acoustic detectors configured to detect an acoustic wave generated from an object irradiated with light from the light source and output an electric signal, the plurality of acoustic detectors including a first acoustic detector having a first frequency sensitivity characteristics to output a first electric signal and a second acoustic detector having a second frequency sensitivity characteristics, which is different from the first frequency sensitivity characteristics, to output a second electric signal;
a memory configured to hold a correction function created based on a third frequency sensitivity characteristics obtained based on the first and the second frequency sensitivity characteristics; and
an information processor configured to: (a) read the correction function from the memory, (b) correct the first and second electric signals in accordance with the correction function, thereby acquiring corrected first and second electric signals, and, (c) generate image data representing object information using the corrected first and second electric signals.

2. The apparatus according to claim 1, wherein the memory is configured to hold the correction function, which corresponds to a variation in sensitivity according to a frequency in the third frequency sensitivity characteristics.

3. The apparatus according to claim 2, wherein the memory is configured to hold the correction function created based on a correction standard function having a reduced variation in sensitivity according to a frequency in the third frequency sensitivity characteristics.

4. The apparatus according to claim 3, wherein the memory is configured to hold the correction standard function created by flattening frequency sensitivity characteristics of the third frequency sensitivity characteristics.

5. The apparatus according to claim 3, wherein the memory is configured to hold the correction standard function created by reducing, at a constant rate, an intensity of the sensitivity with respect to a predetermined threshold in a frequency band involving the sensitivity in the third frequency sensitivity characteristics.

6. The apparatus according to claim 1, wherein a frequency band involving the sensitivity in the first frequency sensitivity characteristics partly overlaps a frequency band involving the sensitivity in the second frequency sensitivity characteristics.

7. The apparatus according to claim 1, wherein the information processor is configured to perform the correction by applying the correction function to the electric signals converted into frequency domain signals and generates the image data by image reconstruction using the corrected electric signals.

8. The apparatus according to claim 1, wherein the information processor performs the correction by converting the correction function into a time domain function and applying the resultant function to the electric signals and generates the image data by image reconstruction using the corrected electric signals.

9. The apparatus according to claim 1, further comprising a supporter on which the plurality of acoustic detectors are arranged.

10. An apparatus comprising:
a light source;
a plurality of acoustic detectors configured to detect an acoustic wave generated by an object irradiated with light from the light source and output an electric signal, the plurality of acoustic detectors including a first acoustic detector having a first frequency sensitivity characteristics to output a first electric signal and a second acoustic detector having a second frequency sensitivity characteristics, which is different from the first frequency sensitivity characteristics, to output a second electric signal;
a memory configured to hold a correction image filter created based on a third frequency sensitivity characteristics obtained based on the first and the second frequency sensitivity characteristics; and
an information processor configured to: (a) read the correction image filter from the memory, (b) correct image data based on the first electric signal and image data based on the second electric signal in accordance with the correction image filter, and, (c) generate image data representing object information.

11. The apparatus according to claim 10, wherein the memory is configured to hold the correction image filter which corresponds to a variation in sensitivity according to a frequency in the third frequency sensitivity characteristics.

12. The apparatus according to claim 11, wherein the memory is configured to hold the correction image filter created based on a correction standard function having a reduced variation in sensitivity according to a frequency in the third frequency sensitivity characteristics.

13. The apparatus according to claim 12, wherein the memory is configured to hold the correction standard function created by flattening frequency sensitivity characteristics of the third frequency sensitivity characteristics.

14. The apparatus according to claim 12, wherein the memory is configured to hold the correction standard function created by reducing, at a constant rate, an intensity of the sensitivity with respect to a predetermined threshold in a frequency band involving the sensitivity in the third frequency sensitivity characteristics.

15. The apparatus according to claim 10, wherein a frequency band involving the sensitivity in the first frequency sensitivity characteristics partly overlaps a frequency band involving the sensitivity in the second frequency sensitivity characteristics.

16. The apparatus according to claim 10, wherein the information processor is configured to perform the correction by performing image reconstruction on the first and the second electric signals to generate the image data, converting the image data into frequency domain data, and applying the correction image filter to the resultant image data.

17. The apparatus according to claim 10, wherein the information processor is configured to perform the correction by performing image reconstruction on the first and second electric signals to generate the image data, converting the correction image filter into time domain filter, and applying the resultant filter to the image data.

18. The apparatus according to claim 10, further comprising a supporter on which the plurality of acoustic detectors are arranged.
